(19) United States
(12) Patent Application Publication
(10) Pub. No.: US 2022/0023154 A1
(43) Pub. Date: Jan. 27, 2022

(54) Emesis Containment Device

(71) Applicant: Robert S. Gold, Newburgh, IN (US)

(72) Inventor: Robert S. Gold, Newburgh, IN (US)

(21) Appl. No.: 17/385,112

(22) Filed: Jul. 26, 2021

Related U.S. Application Data
(60) Provisional application No. 63/056,585, filed on Jul. 25, 2020.

(51) Int. Cl. A61J 19/00; A61M 1/00

(52) U.S. Cl. CPC A61J 19/00; A61M 2205/75; A61M 1/64

(57) ABSTRACT

An emesis containment device is configured to draw in airborne infectious and other particles and may include a ring guard, emesis bag, filter, and pump to draw air through the filter.

### RELATED APPLICATION

This application claims the benefit of U.S. provisional patent application Ser. 63/056,585, filed Jul. 25, 2020, by Robert S. Gold, and titled “An Emesis Containment Device,” the disclosure of which is hereby incorporated by reference.

### TECHNICAL FIELD

The present application relates to a device that collects emesis.

### BACKGROUND AND SUMMARY OF THE DISCLOSURE

This section introduces aspects that may help facilitate a better understanding of the disclosure. Accordingly, these statements are to be read in this light and are not to be understood as admissions about what is or is not prior art.

An emesis containment device is a product that individuals can use to collect and/or dispose of emesis. One purpose of the device is to reduce the likelihood of emesis contaminating an area or surface. If emesis is not properly contained or cleaned, an individual who is infected with norovirus, other disease, etc. can inadvertently release infectious particles into the air where they can contaminate surfaces or infect other individuals directly. Emesis containment devices uses bags, basins, buckets, etc. to collect and/or retain emesis to be disposed. With some devices, emesis and the infectious particles within are not totally contained and can be released into the air during or after emesis expulsion, increasing the risk of infection for other individuals. The present device seeks to reduce the release of emesis particles by creating a negative airflow around the site of emesis expulsion, reducing the likelihood that infectious particles are released into the surrounding area.

### BRIEF DESCRIPTION OF THE DRAWINGS

The previously described aspects of the disclosure will grow to be appreciated at a greater level once references to the following accompanying illustrations are expounded upon.

FIG. 1 is an image of a user vomiting into an emesis containment device;

FIG. 2 is a detailed exploded view of the emesis containment device containing an emesis bag, a filter, a ring guard with holes, and a housing configured to contain a motor, a fan, and a battery;

FIG. 3 is a side view of the assembled emesis containment device, with the emesis bag in a rolled-up state;

FIG. 4 is an image of the emesis bag in a fully expanded state,

FIG. 5 is a perspective view of the ring guard with holes;

FIG. 6 is a top view of the ring guard with holes;

FIG. 7 is a side view of the ring guard with holes;

FIG. 8 is a top view of the filter positionable into the ring guard;

FIG. 9 is a side view of the filter positionable into the ring guard;

FIG. 10 is a cross-sectional view of the ring guard along line 10-10 of FIG. 6 showing the filter inside the ring guard; and

FIG. 11 is a perspective view of the housing that couples to the ring guard.

### DETAILED DESCRIPTION

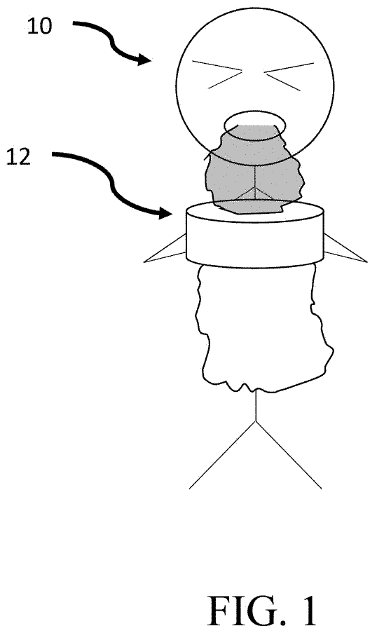

As shown in FIG. 1, a user 10 is vomiting into an emesis containment device 12. Emesis containment device 12 pulls in air from the surrounding area and filters it to create a negative airflow around the site of emesis. The negative airflow and filtration reduces the likelihood that, infectious emesis particles escape into the surrounding area during an emesis episode. After user 10 vomits into device 12, emesis inside device 12 can be disposed. Device 12 can be held by user 10, another individual, or an apparatus during expulsion of emesis by user 10. According to the present disclosure, emesis device 12 can be positioned over the mouth of user 10 or in front of user 10 during emesis expulsion. According to the present disclosure, the exterior of emesis device 12 is made of plastic.

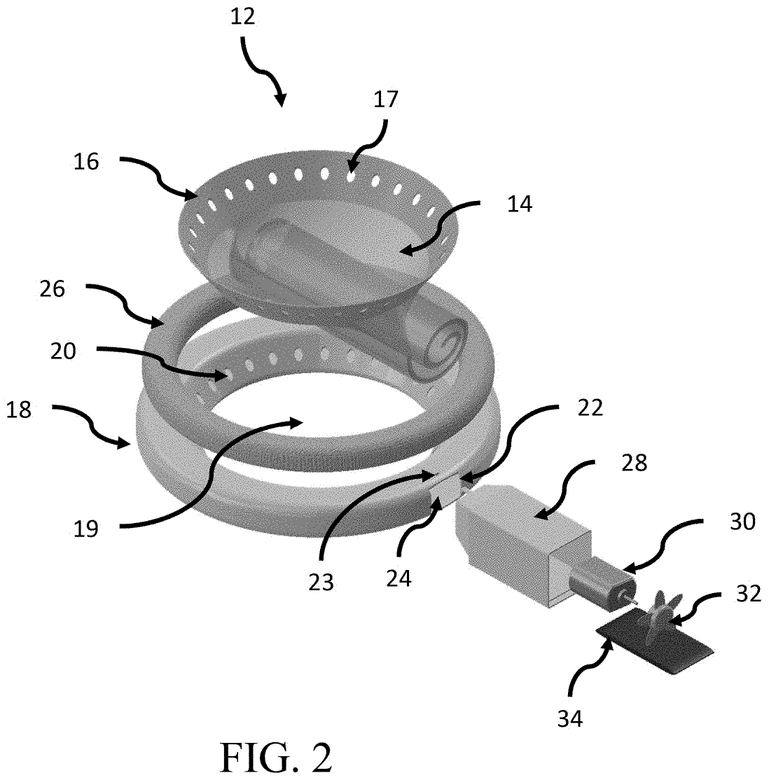

As shown in FIG. 2, emesis device 12 comprises a ring guard/body 18 with holes 20, an air filter 26, an emesis bag 14 with a top ring 16 and holes 17, a housing 28, a motor 30, a fan 32, a battery 34. Fan 32 under the power of motor 30 and battery 34 creates a negative flow of air, pulling air into ring guard 18 and through filter 26. Airborne infectious and other particles resulting from an emesis event are pulled into filter 26 to decrease the likelihood of these particles spreading to the surrounding area.

Housing 28 contains motor 30, fan 32, and battery 34 and cooperates with ring guard 18 to define a removable connection 22 between housing 28 and ring guard 18. The cooperation between ring guard 18 and connection 22 is secured by indents 23 on ring guard 18. Emesis bag 14 rests within a circular opening 19 of ring guard 18. Top ring 16 of emesis bag 14 is larger than opening 19 of ring guard 18 and reduces the likelihood of emesis bag 14 falling through opening 19 of ring guard 18. Filter 26 fits within an interior 24 of ring guard 18 where the air flows through holes 20 towards filter 26.

Ring guard 18 is coupled to housing 28 by a removable connection 22. Housing 28 holds motor 30, fan 32, and battery 34, which provides emesis device 12 with electrical power to generate the negative airflow using motor 30 and fan 32.

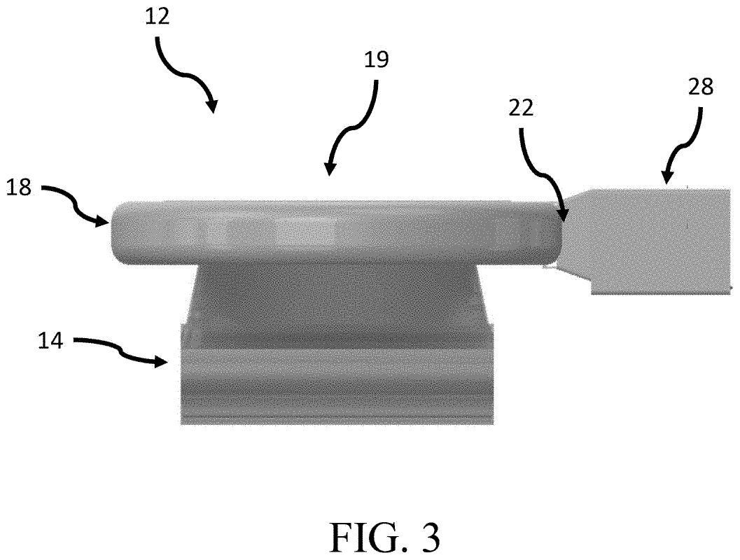

As shown in FIG. 3, emesis bag 14 is in a rolled-up state, positioned within and under ring guard 18. Housing 28 is coupled to ring guard 18 at connection point 22. According to the present disclosure, emesis bag 14 can be expanded from its rolled position to collect emesis in the event of expulsion. Emesis bag 14 can additionally be removed from, emesis containment device 12 by lifting bag 14 vertically through opening 19 of ring guard 18.

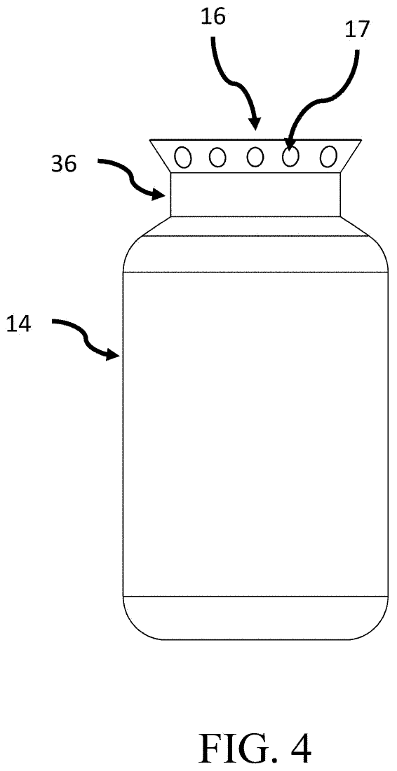

As shown in FIG. 4, emesis bag 14 is fully expanded includes a neck 36 attached to top ring 16. According to the present disclosure, neck 36 of emesis bag 14 is made of plastic and is connected to rim 16. According to the present disclosure rim 16 is also made of plastic and allows emesis bag 14 to sit inside opening 19 of ring guard 18. Rim 16 has holes 17 which align with holes 20 on ring guard 18 to allow air to be drawn through holes 20 during use of emesis collection device 12.

To collect and dispose of emesis with emesis bag 14, user 10 will vomit into emesis containment device 12. The emesis expelled into device 12 will be collected in emesis bag 14. Once user 10 has finished vomiting, the user or care provider will grab emesis bag 14 and spin bag 14 while holding onto ring guard 18. Once a sufficient portion of emesis bag 14 has been twisted, user 10 or a care provider can remove emesis bag 14 from emesis containment device 12 by lifting bag 14 vertically through opening 19 of ring guard 18. User 10 can then tie a knot with the twisted bag 14 or seal emesis bag 14 through alternative means such as clips, twists, etc. before disposing of emesis bag 14. Once properly disposed of, a replacement emesis bag 14 is placed inside opening 19 of ring guard 18. Emesis collection device 12 can then be reused in the same manner to collect and dispose of emesis.

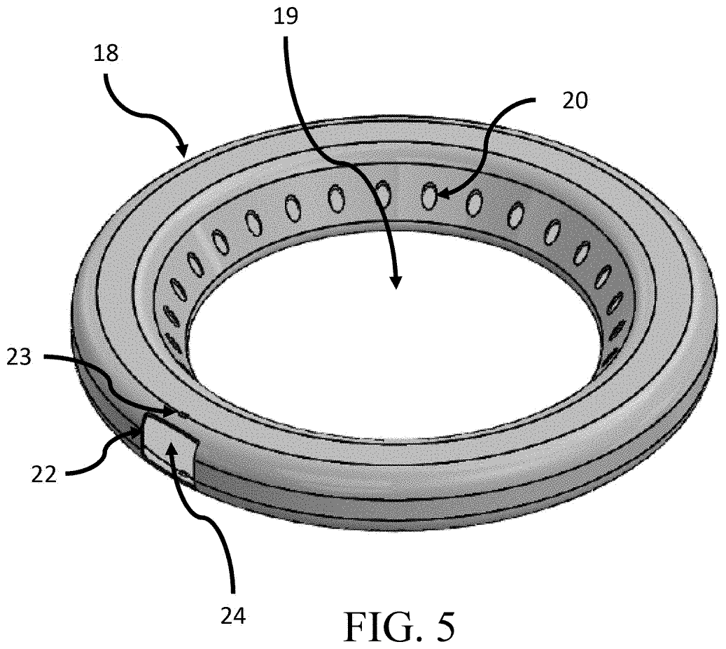

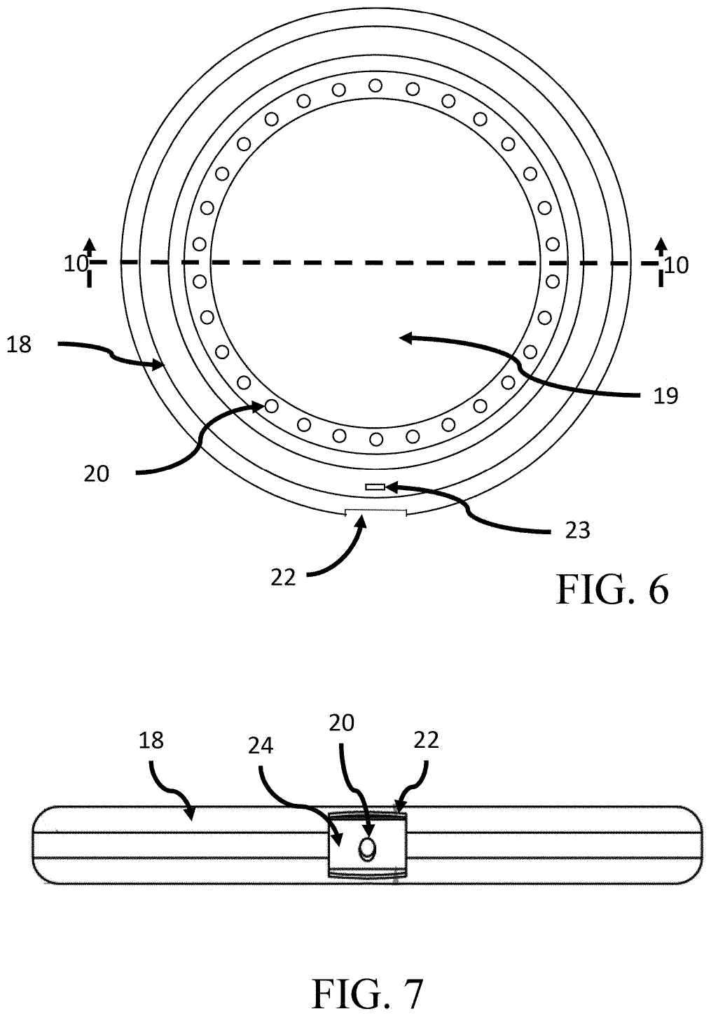

According to the present disclosure as shown in FIGS. 5-7, ring guard 18 is reusable and can be made with plastic material. Opening 19 provides a place for emesis bag 14 to sit inside ring guard 18. Holes 20 on ring guard 18 allow air to flow therethrough toward filter 26 positioned in interior 24 of ring guard 18. Ring guard 18 is attached to housing 28 by a connection 22. The coupling of housing 28 at connection 22 is facilitated by indents 23.

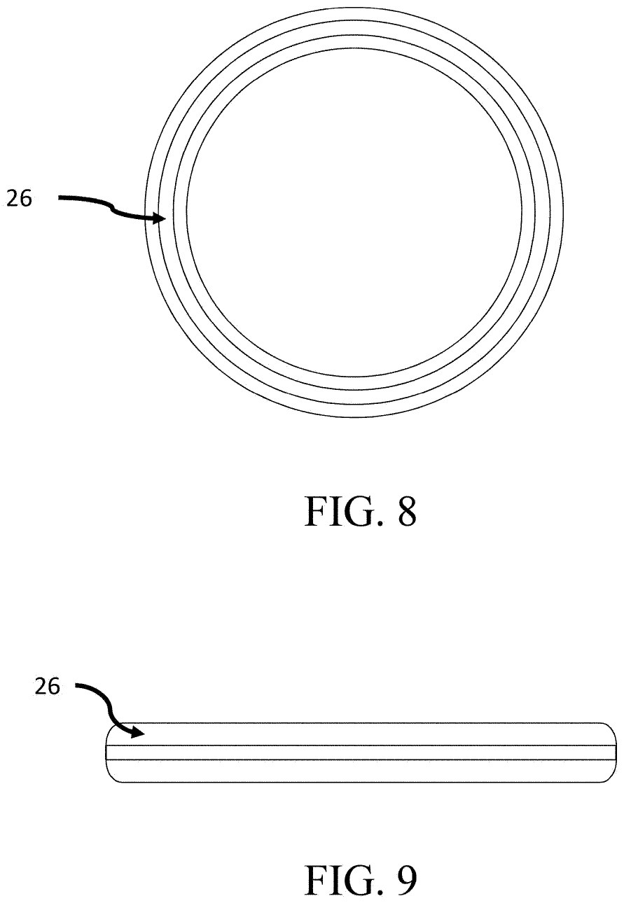

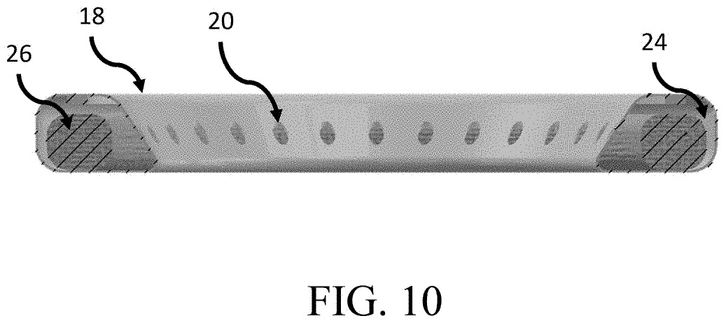

Filter 26 is sized and shaped to be positioned within ring guard 18 as shown in FIGS. 8-10. The material for filter 26 is such that it can block infectious emesis particles and simultaneously let air pass through device 12. According to one embodiment, ring guard 18 may be opened to allow replacement of filter 26.

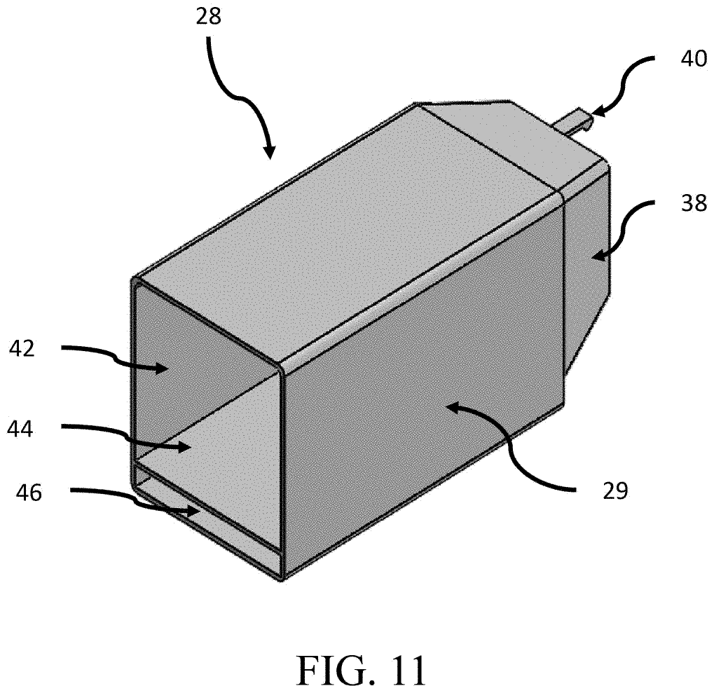

As shown in FIG. 11, housing 28 comprises a housing body 29, a tapered end 38, an attachment clip 40, a hollow interior 42, a fan compartment 44, and a battery compartment 46. Tapered end 38 is positioned at the rear end of housing 28 and is attached to housing body 29. Tapered end 38 has a square opening at its end, allowing hollow interior 42 to extend through the length of housing 28. In the square opening created by tapered end 38, two attachment clips 40 are connected to tapered end 38. One is located at the top of tapered end 38 and the other is located at the bottom. Tapered end 38 of housing body 29 fits over connection point 22 of ring guard 18 and interacts with indents 23. Attachment clips 40 on tapered end 38 facilitate coupling of housing 28 to ring guard 18 by securely clicking into indents 23. Each attachment clip 40 has a corresponding indent 23, for a total of two indents 23. According to the present disclosure, indents 23 are rectangular to accommodate the shape of attachment dips 40. According to the present disclosure housing body 29 is made of plastic. Fan compartment 44 is designed to be large enough to accommodate motor 30 and fan 32. Battery compartment 46 is located directly below fan compartment 46 and is designed to accommodate battery 34 which can be connected to motor 30. According to one embodiment, a switch (not shown) or other device is provided on ring guard 18 to detect user 10 grabbing ring guard 18, staring motor 30 and fan 32.

1. An emesis collection device comprising:
an outer body defining an opening;
an emesis bag aligned with the opening; and
a pump positioned to draw air through the outer body away from the opening.

2. The emesis collection device of claim 1, further comprising an at least one filter enclosed by the outer body.

3. The emesis collection device of claim 2, wherein the at least one filter comprises a material configured to filter particles greater than or equal to 0.20 μm.

4. The emesis collection device of claim 1, wherein the outer body has a surface and a plurality of holes on the surface through which the air is drawn.

5. The emesis collection device of claim 3, wherein the emesis bag has a rim including a plurality of holes that cooperate with the plurality of holes on the surface of the outer body.

6. The emesis collection device of claim 1, wherein the pump comprises:
a fan;
a motor connected to the fan; and
a battery connected to the motor.

7. The emesis collection device of claim 6, wherein the battery is positioned externally of the outer body.

8. An emesis collection device comprising:
a torus-shaped body having an inner surface and a plurality of openings distributed across the inner surface;
a housing connected to the torus-shaped body;
a motor connected to the housing; and
a fan electrically coupled to the motor, the fan configured to pull air towards the inner surface of the torus-shaped body.

9. The emesis collection device of claim 8, further comprising a ring guard coupled to the housing, wherein the ring guard has a plurality of openings distributed across a surface of the ring guard.

10. The emesis collection device of claim 9, wherein the ring guard is configured to attach and detach to the housing.

11. The emesis collection device of claim 8, wherein the housing is configured to attach and detach to the torus-shaped body.

12. The emesis collection device of claim 8, further comprising a bag positioned within the torus-shaped body to receive emesis.

13. The emesis collection device of claim 12, wherein the bag is configured to attach and detach to the torus-shaped body.

14. The emesis collection device of claim 8, further comprising an air filter encompassed within the inner surface of the torus-shaped body.

15. A method of collecting emesis comprising:
providing an emesis collection device including an outer body supporting and at least one bag and at least one bag removable from the emesis collection device;
generating an airflow towards an interior of the at least one bag of the emesis collection device; and
collecting emesis in the at least one bag.

16. The method of collecting emesis of claim 15, wherein air is drawn into the interior of the at least one bag during the generating step.

17. The method of collecting emesis of claim 15, wherein the emesis collection device further comprises a pump generating the airflow towards the interior of the at least one bag of the emesis collection device.

18. The method of collecting emesis of claim 15, wherein the emesis collection device further comprises a filter substantially preventing particles of emesis from being expelled into a surrounding environment.

19. The method of collecting emesis of claim 15, wherein the emesis collection device has an opening defined by an outer body and the at least one bag is aligned with the opening.

20. The method of collecting emesis of claim 15, further comprising a step of removing the bag from the emesis collection device after the step of collecting the emesis.
